(19) United States
(12) Patent Application Publication
(10) Pub. No.: US 2025/0127190 A1
(43) Pub. Date: Apr. 24, 2025

(54) MYCELIAL FOOD SUBSTITUTE AND A METHOD OF ITS PRODUCTION

(71) Applicant: Bumble Be GmbH, Baldham (DE)

(72) Inventor: Guido ALBANESE, Baldham (DE)

(73) Assignee: Bumble Be GmbH, Baldham (DE)

(21) Appl. No.: 18/869,087

(22) PCT Filed: May 25, 2023
(86) PCT No.: PCT/EP2023/064115

§ 371 Date: Nov. 25, 2024

(30) Foreign Application Priority Data
May 25, 2022 (EP) 22175503.6

(51) Int. Cl. A23J 3/22; A23C 20/02; A23J 3/34; A23L 31/00; C12N 1/12; C12N 9/10

(52) U.S. Cl. CPC A23J 3/227; A23C 20/02; A23J 3/34; A23L 31/00; C12N 1/12; C12N 9/1044; C12Y 203/02013

(57) ABSTRACT

The present invention refers to a method for the production of a whole-cut mycelial food substitute having fibrous structure optionally further comprising algae, bacteria, archaea or a combination thereof and a whole-cut mycelial food substitute obtainable by such method. In addition, the invention is directed to the use of a whole-cut mycelial food substitute for the production of a food product as well as such food product comprising an edible.

The present invention refers to a whole-cut mycelial food substitute having fibrous, muscle-like structure, comprising a fungal mycelium and optionally bacteria, algae, archaea, animal cells or a combination thereof, a method for its production and a food product comprising the whole-cut mycelial food substitute.

### TECHNICAL BACKGROUND

Animal protein replacement products are gaining consumer popularity amongst rising concerns on climate change and animal welfare as well as personal health. All of the current commercialized offerings are either plant-based or derived by fermenting plant materials. One of the shortfalls of these animal protein replacement products is their lack of a natural texture similar to the meat or fish products they aim to replace. To overcome this short-fall there are as of now three approaches deployed: one of them is wet or dry extrusion at high temperature and pressure of the protein isolates aided by a texturizing agent such as carboxymethyl cellulose. The second is adding cold- or hot-gelling texturizing agents such as starches, gums or co-agulants such as wheat gluten or albumin (egg white), recently also used in combination with 3-D printing. These first two approaches have disadvantages: While mimicking the texture of meat products works well in extrusion processes, carboxymethyl cellulose has been associated with inflammation of the gut, altering microbiota and detected as a trigger of inflammatory bowel diseases such as ulcerative colitis and Crohn's disease. Starches and gums on the other hand are falling short of creating both firmness and elasticity in the alternative protein products they are applied to. Both groups of texturizers are adding to rising concerns of consumers that alternative protein products contain too many additives making the overall product not healthy. Although wheat gluten and albumin provide better texture properties compared to starches and gums, their disadvantage in meat or fish protein replacement products is that they are allergens and the resulting products are not suitable for consumers including for example vegan consumers.

The third approach to texturize plant-based proteins is to cross-link specific proteins with enzymes. The most common one is the use of transglutaminase, which is linking lysine and glutamine residues bound to proteins or peptides into an isopeptide bond. This approach has been extensively used in Tofu manufacturing.

A specific class of alternative meat and fish replacement products based on fungal mycelium already have a micro-texture provided by the filamentous growth of the mycelium. To obtain moldable and chunky products, Marlow Foods who started marketing these products in the late 1980′ under the brand name Quorn—used a combination of wheat gluten and albumin. In recent years it has started offering vegan products with a combination of gums and starches as well.

In WO 03/007728A2 enzymatic treatment with transglutaminase is applied to fungal protein by shredding the obtained mycelium from Rhizosporus fungi, thus exposing the contained proteins. The shredded cells are then mixed with transglutaminase and were moulded into burger patties.

Similarly in US2020/060310 plant-based proteins which have been subjected to mycelial fermentation are treated with transglutaminase to modify their texture.

However, neither the enzymatic treatment of the shredded cells nor the binding of fungal mycelium with wheat gluten albumin or gums and starches offers the possibility to obtain meat, fish or dairy replacement products in larger bodies, resembling whole-cut filets or in case of dairy cheese slices or blocks.

Thus, the problem underlying the present invention is the provision of a method for the production of improved meat and dairy replacement products. These products fulfil taste expectations of a broad range of consumers including true friends of meat due to the fibrous structure and mouthfeel of a natural meat or dairy product as well as vegetarians and even vegans. The improved texture of these replacement products is based for example on a pressure resistance and/or a cut resistance of the replacement products of the present invention which is corresponding to a natural meat and dairy product. At the same time such meat or dairy replacement products do not have undesirable off-flavors which make consumer hesitant to accept replacement products.

The present invention aims to solve this problem of a meat, fish meat or dairy alternatives having natural mouthfeel, for example due to a firm, cohesive and juicy, respectively, texture for meat and fish, by a food substitute based on mycelium from edible fungi. The present invention provides an improved and simplified method for the production of fungal mycelium based food substitutes. The fungal mycelium based food substitute is for example only produced from mycelium of edible fungi or is for example produced from a combination of the fungal mycelium with bacteria, algae, archaea and/or animal cells. The substitute is for example vegan, vegetarian or includes animal cells. The food substitute offers an improved texture, which results in larger whole-cut, filet-like products imitating the muscle-like structures of meat, fish meat or dairy products such as slices or chunks of cheese without addition of additives like gums or starches. Thus, the food substitute of the present invention leads to an improved consumer acceptance.

### SUMMARY OF THE INVENTION

The method for the production of the food substitute of the present invention to texturize mycelium-based protein products leads to an improved, more complex texture of the food substitute that mimics whole-cut, for example filet-like, meat and fish meat more closely to natural meat, than could be reached with a method of the prior art. This improved texture properties will significantly increase consumer acceptance and open up this raw product for multiple new applications of replacements of fish and meat products. Further, the method for the production of the food substitute of the present invention to texturize mycelium-based protein products leads to improved food substitutes having a clean flavor. Clean flavor is the characteristic flavor of a food product as expected by a consumer. Off-flavors are undesired flavors e.g., caused by binding agents, excessive processing, byproducts etc. Off-flavors for example originate in raw materials, from chemical changes during food processing and storage, from micro-organisms and/or from additives, such as binding agents. Thus, off-flavors are taints in food products which result in reduced quality of the food product and in consequence in poor consumer acceptance.

For dairy products the present invention opens mycelium from edible fungi up to new applications for example to hard or semi-hard cheese types such as edamer or camembert in block form without the need of addition of other texturizers such as gums or starches.

The present invention refers to a method for the production of a whole-cut mycelial food substitute having fibrous structure comprising or consisting of the steps of
a) culturing a fungus in growth medium to form a mycelium,
b) harvesting the mycelium of the fungus for example by filtration, centrifugation, pressing, screening, evaporation or a combination thereof to isolate the mycelium of the fungus from the growth medium,
c) adding a cross-linking enzyme to the harvested mycelium of the fungus, wherein hypha of the mycelium are cross-linked, and
d) optionally bringing the linked hypha in a desired form and/or adding further components such as a flavoring agent after step c).

The fungus is for example selected from the division basidiomycota, ascomycota or a combination thereof. In particular, the fungus is for example Pleurotus pulmonarius, Pleurotus ostreatus such as Pleurotus ostreatus, var. Florida, or a combination thereof.

In step a) an algae, bacteria, archaea, animal cells or a combination thereof is for example cultured together with the fungus; an algae, bacteria, archaea, animal cells or a combination thereof is added to the harvested mycelium of step b); and/or an algae, bacteria, archaea, animal cells or a combination thereof is added to cross-linked hypha of step c). The bacteria is for example selected from the group consisting of acetobacter, arthrobacter, bacillus, bifidobacterium, brachybacterium, brevibacterium, carnobacterium, corynebacterium, enterococcus, gluconacetobacter, gluconobacter, hafnia, halomonas, kocuria, komagataeibacter, lactobacillus, lactococcus, leuconostoc, macrococcus, microbacterium, micrococcus, oenococcus, propionibacterium, staphylococcus, streptococcus, streptomyces, tetragenococcus, weissella, zymomonas or a combination thereof. The algae is for example selected from the division chlorophyta; optionally the algae is Chlorella vulgaris and/or Arthrospira platensis. The animal cell is for example a natural or a cell-culture derived animal cell. The animal cell is for example in single-cell state. The animal cell is for example from bovine, ovine, equine, murine, poultrine, porcine, fish and/or crustacean, such as shrimp or lobster, origin.

The harvested mycelium of step b) is for example brought into a desired form before the cross-linking enzyme is added to the harvested mycelium in step c). The cross-linking enzyme is for example selected from the group consisting of a laccase, peroxidase, tyrosinase, sortase, lysyl oxidase, amine oxidase, transglutaminase or a combination thereof. The cross-linking enzyme is for example dropped or sprayed over the harvested mycelium and optionally the bacteria, algae or a combination thereof. The crosslinking enzyme is for example present in a concentration of about 0.01 to 5%, about 0.1 to 3% or about 1 to 2%.

The method of the present invention optionally comprises in optional step d) further amendments. Such amendments are for example adding of a non-animal fat, flavoring agent, coloring, preservative, texture modifier, a processing aid or a combination thereof. One or more of these amendments are performed on the harvested mycelium before cross-linking of the hypha of the mycelium and/or after cross-linking of the hypha of the mycelium. Such amendment is for example adding a non-animal protein after cross-linking of the hypha of the mycelium.

A texture modifier is for example starches derived from, but not limited to, potato, rice, wheat, and/or corn tapioca; gums such as, but not limited to, locust bean gum, carrageenan or other algae derived texturizers. A processing aid is for example an enzyme such as TGase, liquid nitrogen, ozone, antifoam agents, brine and/or caustic soda.

The present invention further refers to a whole-cut mycelial food substitute having fibrous structure obtainable by a method of the present invention. The whole-cut mycelial food produced by a method of the present invention is for example meat, fish meat or a dairy product such as cheese.

The whole-cut mycelial food substitute of the present invention has for example a fibrous structure comprising or consisting of a fungal mycelium and optionally additionally comprising or consisting of bacteria, algae, archaea or a combination thereof, wherein hypha of the mycelium are cross-linked. The cross-linked hypha form fibers having for example a length of about 1 to 50 mm, about 2 to 30 mm or about 3 to 20 mm.

The whole-cut mycelial food substitute of the present invention has for example a thickness of about 1 to 500 mm, about 3 to 450 mm, about 5 to 400 mm, about 8 to 350 mm, about 10 to 300 mm, about 15 to 250 mm, about 20 to 200 mm, about 30 to 150 mm or about 50 to 100 mm. It has a cut resistance of for example about 250 to about 15000 g or of about 400 to about 5000 g, for example wherein the area is of about 600 to about 30000 g sec or about 800 to about 15000 g sec. It has a water content of for example about 5 to 98%, about 10 to 90%, about 15 to 80%, about 20 to 70%, about 30 to 60%, about 40 to 50 or about 5 to 20%.

In addition, the present invention relates to the use of the whole-cut mycelial food substitute of the present invention for the production of a food product. This food product comprises the whole-cut mycelial food substitute and is for example a whole-cut mycelial food product. The food product, for example whole-cut mycelial food product, is for human and/or animal consumption.

Moreover, the present invention is directed to a food product comprising or consisting of a whole-cut mycelial food substitute of the present invention and an edible for example selected from the group consisting of flavoring agents such as spices, colorings, processing aids, conserving aids, plant-based proteins, fats and a combination thereof. The food product of the present invention is for example part of a ready-to-eat meal or is a meal component. The food product of the present invention is for example breaded, fried, cooked, dried or a combination thereof or is for being breaded, fried, cooked, dried or a combination thereof.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DETAILED DESCRIPTION

The present invention refers to a method for the production of a mycelial food substitute, preferably a whole-cut mycelial food substitute, such as meat, fish meat or dairy products having fibrous structure and for example providing muscle-like texture. The pressure resistance and the cut resistance, respectively, whole-cut mycelial food substitute corresponds to the pressure resistance and/or the cut resistance of a natural meat, fish meat or dairy product such as cheese. The muscle-like texture of the whole-cut mycelial food substitute is supported by enzymatic cross-linking the hypha of the mycelium. The cross-linking enzyme is for example selected from the group consisting of laccase, peroxidase, tyrosinase, sortase, lysyl oxidase, amine oxidase, transglutaminase or a combination thereof.

The cross-linking enzyme is present in the food substitute for example in a concentration of about 0.01 to 5%, about 0.1 to 3%, or about 1 to 2%. The enzyme is for example used as a process aid.

The texture of former meat, fish meat or dairy alternative products known from the prior art has already been influenced by the use of transglutaminase (TGase). In the former products, the administration of TGase results in covalent cross-linking between the proteins glutenin and gliadin. Specifically, TGase catalyzes the reaction between the amine (NH₂) groups of glutamine and lysine to form covalent ε-(γ-glutamyl)lysine bridges (G-L bonds), which are not affected by processing (cooking) or the food matrix environment. Examples of previous TGase applications are crab-imitation products such as surimi or soy-based based products, where it is used directly or on their protein isolates such as in Tofu to increase their firmness. All of these texture modifications rely on freely available terminal amine (NH₂) groups of glutamine and lysine, which are made available to the TGase by disrupting the cellular structure of the animal, plant- or fungi-based raw materials such as fungal mycelium or by providing directly exogenous protein material to the product prior addition of the enzyme.

Surprisingly in the present invention it has been found that fungal mycelium can be transformed into whole-cut, filet-like meat or fish meat having a muscle-like structure, or dairy products such as cheese slices or blocks, all having a chewy texture, without disrupting the cell structure of the mycelium, in particular the hypha of the mycelium through mechanical stress or addition of exogenous proteins. Thus, the method for the production of whole-cut mycelial food substitute according to the present invention is simplified and renders exposing fungal proteins by a disruption of the mycelium and the hypha, respectively, and/or adding exogeneous proteins and protein sources, respectively, such as gluten, prior cross-linking superfluous. Moreover, it has been surprisingly found that the method for the production of the food substitute of the present invention does not cause the occurrence of any undesirable off-flavors. The texture of the mycelial food substitute of the present invention is gained by adding a cross-linking enzyme to the harvested mycelium of the fungus. This means that no mechanical and/or chemical pre-treatment for enriching proteins and/or releasing naturally contained proteins is required prior to adding a cross-linking enzyme. For example, a cross-linking enzyme is added directly after harvesting the fungal mycelium. Further, the mycelial food substitute of the present invention is gained without extrusion such as wet or dry high-pressure extrusion to produce a texturized protein. The mycelial food substitute of the present invention has for example a chewy texture.

Formerly, the fungal mycelium was first separated (e.g. via filtration) from the culture medium and then mechanically cut into smaller pieces (e.g. with a food processor) exposing the proteins contained in the cells. Afterwards, the macerated mycelium was mixed with TGase and finally mold into a desired shape such as a burger patty.

Fungal mycelium of the present invention is for example grown in sub-merged fermentation, co-fermentation (described, e.g., in EP21173540.2) or solid-state fermentation. After completing the fermentation the fungal mycelium is separated from any liquid by means for example by filtration, centrifugation, pressing, screening, evaporation or a combination thereof.

In the present invention, the fungal mycelium is for example directly treated with the enzyme such as the TGase after separation of the fungal mycelium from the culture medium. The TGase is administered to the mycelium either before and/or after the mycelium is brought into the desired shape. The enzyme such as the TGase is for example dissolved in a liquid such as water and dropped or sprayed over the fungal mycelium separated from the culture medium.

In the present invention, no additional accumulation of proteins is required. Neither disrupting the fungal mycelium for exposing fungal proteins nor adding exogenous proteins or protein sources, such as gluten, is required prior to adding a crosslinking enzyme to the fungal mycelium in order to facilitate cross-linking of the fungal mycelium. The method for production of the present invention facilitates to transform the fungal mycelium into whole-cut, filet-like meat or fish meat having a muscle-like structure, or dairy products such as cheese slices or blocks without additional accumulation and/or addition of proteins. The texture of and thus, the whole-cut mycelial food substitute is purely formed by the harvested fungal mycelium which is cross-linked by a cross-linking enzyme added to the fungal mycelium. No chemical and/or mechanical protein enrichment is performed prior to adding a cross-linking enzyme to the harvested fungal mycelium. The method for the production of a whole-cut mycelial food substitute according to the present invention comprises directly adding a cross-linking enzyme after harvesting the fungal mycelium is performed. Hence, the mycelium is not pretreated before addition of the cross-linking enzyme.

The fungal mycelium can be further mixed with an algae such as microalgae, bacteria such as lactic acid bacteria, archaea, animal cells, such as a single cell of chicken, pork, beef, salmon or tuna origin, or a combination thereof before and/or after TGase is administered. Alternatively, the fungal mycelium, bacteria, archaea, algae and/or animal cells are separately treated with TGase and are combined after the TGase treatment. Thus, also the bacteria, archaea, algae and/or the animal cells are not mechanically treated to disrupt the cells. Also these mixtures of the present invention are characterized by a chewy texture.

The mycelial substitute can further be characterized by a secondary structure. The secondary structure is for example layers, tubes, laminates and/or cavities. The secondary structure for example mimics adjacent muscle strands, muscle tissue, connective tissue, fat tissue and/or tendrils, e.g., in fish filets. The secondary structure is for example produced by varying the concentration of the cross-linking enzyme in the different parts of the products. For example the secondary structure is produced by selectively deactivating the cross-linking enzyme with short heat treatments, washing the cross-linking enzyme off and/or deactivating the cross-linking enzyme through base or acid treatment.

After addition of the cross-linking enzyme the shaped forms are for example either first packaged or directly stored at 0.5-45° C., preferably between 4-7° C. for 2-48 hours, preferably between 12-24 hours. The pH of the product is for example maintained between 4-10, preferably 6-8 at a level suitable for enzymatic activity as known to the skilled person.

Advantageously, the whole-cut mycelial food substitute of the present invention is produced from edible fungi with an enzyme such as TGase and the specific meat- and cheese-like texture is reached without binding agents such as gums or starches. Thus, the texture is based purely on the cross-linked mycelium. Hence, no binding agents are required in the present invention to obtain elastic and moldable final products such as meat, fish meat such as meat or fish filets or other seafood products such as crustaceans or mollusks, or dairy products such as cheese.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention. The term “about” indicates the variation of a concrete figure of +/−10%. The terms “texturizer”, “texture modifier” and “texturizing agent”, respectively, are used herein interchangeably.

All of the above embodiments deliver a superior, more complex texture to the resulting food substitute and food product comprising the food substitute than the presently existing substitutes and products, respectively. Thus, the texture of the food substitute and food product of the present invention corresponds to the natural product which has not been reached so far by substitutes and products of the prior art. The resulting food substitute and food product comprising the food substitute of the present invention retains it pure original flavor and is for example not impacted by any off-flavors, e.g., resulting from additives or excessive processing. The resulting food substitute and food product comprising the food substitute of the present invention has an improved consumer acceptance.

Texture is about sensory interpretation and refers to those qualities of a food that is felt with for example the fingers, tongue, palate, and/or teeth. Texture is for example defined by the physical force in Newton required in absolute terms or over a period of time in horizontal and vertical dimension to disintegrate the structure of a food material. More subjective measures include for example the surface sensation created with tactile organs such as fingers or tongue which can be qualitatively described with adjectives such as smooth, coarse, sandy etc. The mycelial food substitute of the present invention has for example a chewy texture.

The chewy texture of the food substitute is based on the bite resistance as well as the tear resistance. Bite resistance is not only generated by the higher pressure resistance caused by the enzymatic cross-linking of the mycelium, i.e., for example the hypha of the mycelium, but is essentially influenced by the tear resistance. The tear resistance of the food substitute of the present invention is based on longer, intact hypha of the fungal mycelium in food substitute and food product, respectively. This is reached by avoiding disruption of the fungal mycelium and optionally bacteria and/or algae after separation from the culture medium. The tear resistance of the food substitute and/or the food product of the present invention is characterized by an increased, i.e., a higher cut resistance.

The bite and tear resistance of the food substitute and/or the food product of the present invention is characterized by a higher cut resistance. As an instrumental measure for the complex texture sensation when muscle tissue is torn into smaller pieces by the frontal teeth as bite and tear resistance and then grinded down by the molar teeth as an expression of chewiness is the cut resistance of a texture analyzer.

The texture and/or cut resistance analyzed by a texture analyzer is for example measured as maximum cutting force required to cut the whole-cut mycelial food substitute and for example exerted in the unit of g. Texture properties such as chewiness are for example described by the force exerted over time and is for example exerted in the unit of per second. The texture and/or cut resistance is for example taken as an index of firmness, toughness or fibrousness of the whole-cut mycelial food substitute. The texture and/or cut resistance is for example measured as area. Area is for example defined as total g exerted over a period of time. The area represents the graphical area determined by the x-axis of time in seconds and y-axis as g, where the texture analysis is performed.

The texture, texture properties and/or cut resistance of the food substitute and/or the food product of the present invention is for example analyzed/measured according to any methods of the state of the art, for example by sensory and/or instrumental methods (see e.g., Novakovićand and Tomašević; 59th International Meat Industry Conference MEATCON, 2017, doi:10.1088/1755-1315/85/1/012063; Cheng et al., Comprehensive Reviews in Food Science and Food Safety, Vol. 13, 2014, doi: 10.1111/1541-4337.12043; Lee et al., Journal of Food Science, 1978, Vol 43, No. 5). For example the texture and/or cut resistance of the food substitute and/or the food product of the present invention is measured according to Warner-Bratzler shear force (WBSF) method, Kramer shear compression method and/or according to the texture profile analysis (TPA).

The mycelium used in the present invention originates from at least one fungal strain, bacterial strain or a combination thereof. The mycelium is derived from heterotrophic organisms requiring organic material and oxygen for growth. The mycelium is the vegetative part of a fungus or fungus like bacterial colony, consisting of a mass of branching cells. As such it is an extension of the hypha of fungi that emanates from a fungal spore. The fungus is for example a filamentous fungus.

The fungus is for example selected from the phylum Basidiomycota, Ascomycota, Glomeromycota, Mucoromycota, Zoopagomycota or a combination thereof. Further, the fungus is for example selected from the division agaricomycotina, ustilagomycotina, pezizomycotina, saccharomycotina, taphrinomycetes, diversisporalis, archaeosporales, paraglomerales, endogonales, mucorales, mortieralles, entomophthoromycotina, asellariales, kickxellales, dimargaritales, harpellales, zoopagomycotina, or a combination thereof.

Moreover, the fungus is for example selected from the class tremellomycetes, dacrymycetes, agaricomycetes, exobasisiomycetes, ustilaginomycetes, malasseziomycetes, moniliellomycetes, arthoniomycetes, coniocybomycetes, dothideomycetes, eurotiomyctes, geoglossomycetes, laboulbeniomycetes, lecanoromycetes, leotiomycetes, lichinomycetes, orbiliomycetes, pezizomycetes, sordariomycetes, xylonomycetes, or a combination thereof.

Furthermore, the fungus is for example selected from the order filobasidiales, agaricales, amylocorticiales, atheliales, boletales, jaapiales, lepidostromatales, geastrales, gomphales, hysterangiales, phallales, auriculariales, cantherellales, corticiales, gleophylalles, hymenochaetales, polyporales, russulales, sebacinales, stereopsidales, thelephorales, trechisporales, ceraceosorales, doassansiales, entyomatales, exobasidiales, georgefischeriales, microstromatales, tilletiales, urocystales, ustilaginales, malassezioales, moniliellales, saccharomycetales, coronophorales, glomeralles, Hypocreales, melanosporales, microascales, boliniales, calosphaeriales, chaetospheriales, coniochaetales, diasporthales, magnaporthales, ophiostomatales, sordariales, xylariales, koralionastetales, lulworthiales, meliolales, phylachoralles, trichosphariales, eurotiales, chaetothyriales, pyrenulales, verrucariales, onygenales, mortierellales, mucorales, endogonales, or combinations thereof.

Alternatively, the fungus is selected from the family Filobasidium, Dacromycetaceae, Agaricaceae, Amanitaceae, Bolbitiaceae, Broomeiceae, Chromocyphellaceae, Clavariaceae, Cortinariaceae, Cyphellaceae, Enolomataceae, Fistulinaceae, Himigasteraceae, Hydnangiaceae, Hygrophoraceae, Inocybaceae, Limnoperdacea, Lyophyllaceae, Marasmiaceae, Mycenacea, Niaceae, Pellorinaceae, Physalacriaceae, Pleurotacea, Pluteaceae, Porotheleaceae, Psathyrellaceae, Pterulacea, Schizophyllaceae, Stephanosporaceae, Strophariaceae, Tricholomataceae, Typhulaceae, Boletaceae, Boletinellaceae, Coniophoraceae, Diplocystaceae, Gasterellaceae, Gastrosporiaceae, Gomphidiaceae, Gyroporaceae, Hygrophoropsidaceae, Paxillaceae, Protogastraceae, Rhizopogonaceae, Sclerodermataceae, Serpulaceae, Suillaceae, Tapinellaceae, Hymenochaetaceae, Repetobasidiaceae, Schizoporaceae, Cystostereaceae, Fomitopsidaceae, Fragiporiaceae, Ganodermataceae, Gelatoporaceae, Meripilaceae, Merulaciaea, Phenerochaetaceae, Polyporaceae, Sparassidaceae, Steccherinaceae, Xenasmataceae, Albatrellaceae, Amylostereaceae, Auriscalpaceae, Bondarzewiaceae, Echinodontiaceae, Hericiaceae, Hybogasteraceae, Lachnocladiaceae, Peniphoraceae, Russulaceae, Gloeocyctidiellacceae, Stereaceae, Ustilaginomycetes, Saccharomycetaceae, Saccharomycodaceae, Saccharomycopsidaceae, Chaetomiaceae, Lasiosphaeriaceae, Sordariaceae, or a combination thereof.

Further, the fungus is selected from the genus Neurospora, Aspergillus, Trichoderma, Pleurotus, Ganoderma, Inonotus, Cordyceps, Ustilago, Rhizopus, Tuber, Fusarium, Pennicillium, Xylaria, Trametes, or a combination thereof.

Furthermore, the fungus is selected from the group consisting of Aspergillus oryzae, Rhizopus oryzae, Fusarium graminareum, Cordyceps militaris, Cordyceps sinensis, Tuber melanosporum, Tuber magnatum, Pennicillium camemberti, Neurospora intermedia, Neurospora sitophila, Xylaria hypoxion, or a combination thereof.

If the division of Basidiomycota is selected, the preferred subdivision is Agaromycotina. If the subdivision Agaromycotina is selected, the preferred class is Agaricomycetes. If the class Agaricomycetes is selected, the order Agaricales, Auriculariales, Boletales, Cantharellales, Polyporales and Russulales are preferred. If the order Boletales is selected, the families Boletaceae and Sclerodermataceae are preferred. If the order Cantharellales is selected, the families Cantharellaceae and Hydnaceae are preferred. If the order Agaricales is selected, the families Agaricaceae, Fistulinaceae, Lyophyllaceae, Marasmiaceae, Omphalotaceae, Physalacriaceae, Pleurotaceae, Schizophyllaceae, Strophariaceae, and Tricholomataceae are preferred.

If the order Polyporales is selected, the families Ganodermataceae, Meripilaceae, Polyporaceae, and Sparassidaceae are preferred. If the order Russulales is selected, the families Bondarzewiaceae and Hericiaceae are preferred. If the order Auriculariales is selected, the family Auriculariaceae is preferred. If the family Pleurotaceae is selected, the genus Pleurotus is preferred and most preferred is the species Sapidus. If the division Ascomycota is selected, the subdivision Pezizomycotina is preferred. classes Pezizomycetes and If the subdivision Pezizomycotina is selected, the Sordariomycetes are preferred. If the class Pezizomycetes is selected, the order Pezizales is preferred. If the order Pezizales is selected, the families Morchellaceae and Tuberaceae are preferred.

If the class Sordariomycetes is selected, the order Hypocreales and Sordariales are preferred. If the order Hypocreales is selected, the families Cordycipitaceae and Nectriaceae are preferred. If the order Sordariales is selected, the family Sordariaceae is preferred.

Bacteria used in the present invention are for example bacteria typically used in food preparation. The bacteria encompass for example the families of acetobacter, arthrobacter, bacillus, bifidobacterium, brachybacterium, brevibacterium, carnobacterium, corynebacterium, enterococcus, gluconacetobacter, gluconobacter, hafnia, halomonas, kocuria, komagataeibacter, lactobacillus, lactococcus, leuconostoc, macrococcus, microbacterium, micrococcus, oenococcus, propionibacterium, staphylococcus, streptococcus, streptomyces, tetragenococcus, weissella, zymomonas and a combination thereof.

Archaea used in the present invention are for example bacteria typically used in food preparation. The archaea encompass for example Aenigmarchaeota, Nanohaloarchaea, Huberarchaeaota, Nanoarchaeota, Undinarchaeota, Iainarchaeota, Micrarchaeota, Altiarchaeota, Euryarchaeida, Proteoarchaeota, and a combination thereof. Euryarchaeida comprise for example Hydrothermarchaeota, Hadarchaeota, Methanobacteriota, Thermoplasmatota, Halobacteriota and a combination thereof. Methanobacteriota encompass for example Acherontia such as Thermococci, Methanomada such as Methanopyri, Methanococci, Methanobacteria, or a combination thereof. Thermoplasmatota comprise for example Izemarchaea, Pontarchaea, Poseidoniia, Thermoplasmata or a combination thereof. Halobacteriota comprise for example Methanonatronarchaeia, Archaeoglobia, Methanoliparia, Methermicoccus, Syntropharchaeia, Methanocellia, Methanosarcinia, Methanomicrobia, Halobacteria or a combination thereof. Proteoarchaeota encompass for example Thermoproteota such as Korarchaeia, Bathyarchaeia, Nitrososphaeria, Methanomethylicia, and/or Thermoproteia, Asgardaeota such as Lokiarchaeia, Thoarchaeia, Heimdallarchaeia or a combination thereof.

Algae such as microalgae used in the present invention are for example grown under photoautotrophic, heterotrophic or mixotrophic conditions.

If the algae is selected from the division Chlorophyta the class of Trebouxiophyceae, Chlorodentrophyceae and Chlorophyceae are preferred. If the class of Trebouxiophyceae is selected, the order Chlorellales is preferred. If the order Chlorellales is selected, the family Chlorellaceae is preferred. If the family Chlorellaceae is selected, the genus Chlorella is preferred and most preferred is the species Vulgaris. If the class of Chlorophyceae is selected, the order Chlamydomonadales and Sphaeropleales are preferred. If the order Chlamydomonadales is selected, the families of Haematococaceae, Dunaliellaceae and Chlamydomonadaceae are preferred. If the family Chlamydomonadaceae is selected, the genus Chlamydomonas is preferred and most preferred is the species Reinhardtii. If the family of Haematococaceae is selected, the genus Haematococus is preferred and most preferred is the species Pluvialis. If the family of Dunaliellaceae is selected, the genus Dunaliella is preferred and most preferred is the species Salina. If the order Sphaeropleales is selected, the family of Scenedesmaceae is preferred. If the family Scenedesmaceae is selected, the genus Scenedesmus is preferred and most preferred are the species Dimorphus and Acuminatus. If the class of Chlorodentrophyceae is selected, the order Chlorodendrales is preferred. If the order Chlorodendrales is selected, the family Chlorodendraceae is preferred. If the family Chlorodendraceae is selected, the genus Tetraselmis is preferred and most preferred are the species Suecica and Striatis. If the microalgae are selected from the division cyanobacteria, the class of Cyanophyceae is preferred. Within the class of Cyanophyceae, the order Oscillatoriales and Synechococcales are preferred. If the order Oscillatoriales is selected, the family Microcoleaceae is preferred. If the family Microcoleaceae is selected, the genus Arthrospira is preferred and most preferred is the species Platensis. If the order Synechococcales is selected, the family Synechococcaceae is preferred. If the family Synechococcaceae is selected, the genus Synechococcus is preferred. If the microalgae are selected from the division Haptophyta, the class of Prymnesiophyceae and Pavlovophyceaea are preferred. If the class of Prymnesiophyceae is selected, the order Isochrysidales is preferred. If the order Isochrysidales is selected, the family Isochrysidaceae is preferred. If the family Isochrysidaceae is selected, the genus Isochrysidus is preferred and most preferred is the species Galbana. If the class of Pavlovophyceaea are selected, the order Pavlovales is preferred. If the order Pavlovales is selected, the family Pavlovaceae is preferred. If the microalgae are selected from the division Ochrophyta the class of Bacillariophyceae, Coscinodiscophyceae, Labyrinthulomycetes, Xantophyceae and Eustigmatophyceae are preferred. If the class of Bacillariophyceae is selected, the order Bacillariales is preferred. If the order Bacillariales is selected, the family Bacillariaceae and Phaeodactylaceae are preferred. If the family Bacillariaceae is selected, the genus Nitzschia is preferred and most preferred is the species Dissipata. If the family Phaeodactylaceae is selected, the genus Phaedactylum is preferred and most preferred is the species Tricornutum. If the class of Coscinodiscophyceae is selected, the order Thalassiosirales and Biddulphiineae are preferred. If the order Thalassiosirales is selected, the family Skeletonemataceae and Thalassiosiaceae are preferred. If the family Skeletonemataceae is selected, the genus Skeletonema is preferred. If the family Thalassiosiaceae is selected, the genus Pseudonana is preferred. If the order Biddulphiineae is selected, the family Eupodiscaceae is preferred. If the family Eupodiscaceae is selected, the genus Odontella is preferred and most preferred is the species Aurita. If the class of Labyrinthulomycetes is selected, the order Labyrinthulales and Thraustochytriida are preferred. If the order Thraustochytriida is selected, the family of Thraustochytriidae is preferred. If the order Labyrinthulales is selected, the family Thraustochytriaceae is preferred. If the family Thraustochytriaceae is selected, the genus Schizochytrium is preferred. If the class of Xantophyceae is selected, the order Mischocodcales is preferred. If the order Mischocodcales is selected, the family Pleurochloridaceae is preferred. If the family Pleurochloridaceae is selected, the genus Monodus is preferred and most preferred is the species Subterranea. If the class of Eustigmatophyceae is selected, the order Eustigmatales is preferred. If the order Eustigmatales is selected, the family Monodopsidaceae is preferred. If the family Monodopsidaceae is selected, the genus Nannochloropsis is preferred and most preferred are the species Gaditana, Oceanica and Oculata. If the microalgae are selected from the division Rhodophyta, the class of Porphyridiophyceae is preferred. If the class of Porphyridiophyceae is selected, the order Porphyridiales is preferred. If the order Porphyridiales is selected the family Porphyridiaceae is preferred. If the family Porphyridiaceae is selected, the genus Porphyridium is preferred and most preferred is the species Cruentum.

Animal cell used in the present invention are for example cell culture-derived animal cells. The animal cells used in the present invention are for example in single cell state or an animal cell culture. The animal cell is for example from mammalian origin. The animal cell is for example from bovine, ovine, equine, murine, poultrine, such as duck or chicken and/or porcine origin. The animal cell is for example of fish and/or crustacean origin. The animal cell is for example from bovine, ovine, equine, murine, poultrine, such as duck or chicken, porcine, fish (pisces) origin, such as salmon, tuna, white-fish (cod, pollock, grouper) and/or crustacean, such as shrimp or lobster origin. The animal cell is for example a muscle cell, a fat cell and/or a fibroblast. For example the mycelial food substitute of the present invention comprises 1-90%, for example 5-80%, 10-75%, 15-70%, 20-65%, 25-60%, 30-55%, or 35-50% of animal cells. For example the mycelial food substitute of the present invention comprises at least 1%, at least 5%, at least 10%, at least 20%, at least 30%, at least 40%, at least 45%, at least 50%, at least 60%, at least 70%, at least 80% or at least 90% of animal cells.

For example the mycelial food substitute of the present invention comprises animal cells and algae. For example the mycelial food substitute of the present invention comprises animal cells and microalgae. For example the mycelial food substitute of the present invention comprises 5-95%, 10-90%, 15-80% or 20-70% of fungal mycelium, 1-70%, 5-60%, 10-50% or 15-40% of animal cells and/or 1-60%, 5-50%, 10-40% or 15-30% of algae, such as microalgae. For example the mycelial food substitute of the present invention comprises 5-90% of fungal mycelium, 1-30% of microalgae and/or 1-70% of animal cells. For example the mycelial food substitute of the present invention comprises or consists of Pleurotus spec. such as Pleurotus pulmonarius or Pleurotus ostreatus, e.g., var. Florida or a combination thereof combined with a microalgae of Chlorella spec. such as Chlorella vulgaris and/or animal cells derived from chicken.

For example the mycelial food substitute of the present invention comprises or consists of Pleurotus pulmonarius or Pleurotus ostreatus, e.g., var. Florida or a combination thereof. Pleurotus pulmonarius or Pleurotus ostreatus, e.g., var. Florida or a combination thereof is for example combined with a microalgae such as Chlorella vulgaris and/or Arthrospira platensis. Optionally 20% microalgae Chlorella vulgaris and/or Arthrospira platensis, and 80% mycelium of Pleurotus pulmonarius and/or Pleurotus ostreatus, for example treated with TGase, are co-fermented. Chlorella vulgaris and Arthrospira platensis, or Pleurotus pulmonarius and Pleurotus ostreatus are present in ratios of 10:90, 20:80, 30:70, 40:60, 50:50, 60:40, 70:30, 80:20, or 90:10 of Chlorella vulgaris and Arthrospira platensis, or Pleurotus pulmonarius and Pleurotus ostreatus in the mycelial food substitute.

The mycelial food substitute of the present invention is preferable a whole-cut mycelial food substitute. This means the composition of the mycelial food substitute of the present invention imitates filet-like, fibrous, and/or muscle-like structures and tissue, respectively, of meat, fish meat or dairy products and is grown as homogeneous biological tissue having uniform properties of texture resistance.

Whole-cut is defined as filet, muscle-like, fibrous biological tissue having uniform texture properties such as cut resistance, pressure resistance, extensibility, cohesiveness, adhesiveness, springiness, chewiness, spreadability, viscosity etc. A whole-cut product is for example also referred as a whole-muscle product. A whole-cut product has the appearance of one piece for example comparable to a piece of meat (see e.g., Bihler et al., 2022; Starch; DOI: 10.1002/star.202100157).

The method for the production of the whole-cut mycelial food substitute having fibrous structure comprises or consists of the steps of
a) culturing a fungus in growth medium to form a mycelium,
b) harvesting the mycelium of the fungus for example by filtration, centrifugation, pressing, screening, evaporation or a combination thereof to isolate the mycelium of the fungus from the growth medium,
c) adding a cross-linking enzyme to the harvested mycelium of the fungus, wherein hypha of the mycelium are cross-linked, and
d) optionally bringing the linked hypha in a desired form before or after step c), and/or adding further components such as a flavoring agent, after step c). This component is not a binding agent.

Optionally, an algae, bacteria, archaea, animal cells such as a single cell of chicken, pork, beef, salmon or tuna origin or a combination thereof is cultured together with the fungus in step a). Optionally, in addition or alternatively, an algae, bacteria, archaea or a combination thereof is added to the harvested mycelium of step b). Optionally, in addition or alternatively, an algae, bacteria, archaea or a combination thereof is added to cross-linked hypha of step c).

When the mycelium is harvested in step b), the orientation of the hypha of the mycelium is for example substantially in the same direction, wherein substantially means about 50 to 99%, 70 to 95%, 75 to 90% or 80 to 85% of the hypha of the total mycelium harvested. To reach the same orientation methods such as low pressure extrusion with guiding lenses as deployed in meat processing or 3-D-printers are used. An increasing amount of hypha of the mycelium in the same orientation increases for example the cut resistance.

In addition or alternatively, the cut resistance of the whole-cut mycelial food substitute and the food product comprising the whole-cut mycelial food substitute, respectively, is increased for example by an increase of the amount of TGase, higher temperature, an increase of the ripening time or a combination thereof.

The amount of the TGase is for example calculated as w/w % on filtrate. It varies for example in the range of 0.01 to 5 w/w % or 0.01-0.5 w/w % or 0.1-1 w/w %.

The ripening time of the whole-cut mycelial food substitute or the food product comprising the whole-cut mycelial food substitute depends on the type of product, i.e., meat, fish meat or dairy product; for fresh meat it is for example in the range of 4 to 72 hours, for fresh fish meat or seafood it is for example in the range of 1 to 60 hours, and for a ripened dairy product such as hard cheese it is for example in the range of 5 to 30 days and for ripened meat product such as salami or jerky it is 4-35 days. The times depend on the w/w % amount of TGase added and the storing time used and optionally the applied temperature.

The mycelial food substitute of the present invention for example in form of a meat, fish meat or dairy product has a thickness e.g., in the range of about 1 to 500 mm, about 3 to 450 mm, about 5 to 400 mm, about 8 to 350 mm, about 10 to 300 mm, about 15 to 250 mm, about 20 to 200 mm, about 30 to 150 mm or about 50 to 100 mm.

The mycelial food substitute of the present invention has for example a surface of about 1 to 800 cm², about 3 to 750 cm², about 5 to 700 cm², about 10 to 650 cm², about 15 to 600 cm², about 20 to 550 cm², about 25 to 500 cm², about 30 to 450 cm², about 40 to 400 cm², about 50 to 350 cm², about 75 to 300 cm², about 80 to 250 cm², about 100 to 200 cm², about 1 to 100 cm², about 5 to 75 cm², or about 10 to 50 cm².

The water content of the mycelial food substitute of the present invention is for example about 3 to 98%, about 5 to 95%, about 10 to 90%, about 15 to 80%, about 20 to 75%, about 25 to 70%, about 30 to 60%, about 40 to 50% or 5 to 20%.

The length of the mycelium, i.e., the mycelium fiber in the mycelial food substitute is for example about 1 to 50 mm, about 2 to 45 mm, about 3 to 40 mm, about 4 to 30 mm or about 5 to 20 mm.

The mycelial food substitute has a cut resistance of for example with a force of 250 to 15000 g, preferably 400 to 5000 g and an area of 600 to 30000 g sec, preferably 800 to 15000 g sec.

The mycelia, i.e., the fibers of the mycelium separated from the culture medium are for example oriented in parallel direction. This improves the chewy texture and muscle-like structure of the mycelial substitute of the present invention. The mycelia, i.e., the fibers of the mycelium separated from the culture medium are for example oriented in a fashion where the longer dimension is parallel and adjacent to each other and/or for example combined in strings, which are intertwined with each other. This creates a muscle-like structure, which in turn improves the chewy texture of the mycelial substitute of the present invention

The present invention further refers to a food product comprising a mycelial food substitute, preferably the whole-cut mycelial food substitute, of the present invention and an edible. The food product for example comprises or consists of a whole-cut mycelial food substitute of the present invention and an edible for example selected from the group consisting of flavorings such as spices, colorings, processing aids, conserving aids, plant-based proteins, fats and a combination thereof. The food product of the present invention is for example part of a ready-to-eat meal or is a meal component. The food product of the present invention is for example breaded, fried, cooked, dried or a combination thereof or is for being breaded, fried, cooked, dried or a combination thereof.

The edible is for example selected from the group consisting of flavorings such as spices, colorings, processing aids, conserving aids, plant-based proteins, fats, water-binding additives and a combination thereof.

Food-grade materials such as edibles which are suitable to be combined with the mycelial food substitute of the present invention to form a food product of the present invention. Edibles are for example plant-based proteins, flavoring, coloring agents, carbohydrates, lipids, preservatives and fibers. Plant-based proteins are for example, but are not limited to soy, pea, wheat, rice, lupin, mung bean, potato and chick pea. Flavoring agents are for example herbs and spices or extracts made of them, algae or extracts of them, sauces, which can be potentially fermented such as soy sauce or any other natural, nature-identical or artificial flavoring substances. Coloring agents are for example any food-grade coloring of natural origin such as spice or vegetable extract, represented for e.g. by curcuma or carrot, algae derived coloring agents such as astaxanthin, other natural pigments such as ß-carotene or artificial coloring agents. Carbohydrates are for example, but are not limited to cereals such as corn, wheat or rice and tubers or bloom-tubers such as potato, tapioca and konjac. Lipids can be any plant-derived lipids such as, but not limited to, rapeseed, linseed, coconut, canola, sunflower, olive, algae and palm oil. Preserving agents are for example, but are not limited to, natural acids such as lactic or acetic acid, salt, natural antioxidants such as tocopherol or rosemary extract or artificial preservatives such as potassium sorbate. Fibers from plants that are for example used, but are not limited to, such as coconut, pea, navy beans, algae or fruit pomaces such as apple or citrus.

The edible, exempted a protein, is added to the mycelium separated from the culture medium before or after enzymatic treatment. The protein is added only after the enzymatic treatment for example to avoid any undesired degradation of the added protein influencing disadvantageously the texture of the final product through cross-linking of exogeneous.

The mycelial food substitute in form of a whole-cut meat, seafood or dairy product, for example block dairy product such as hard or soft cheese, or the food product can be further processed by breading, frying, cooking, heating, drying, fermenting, chilling, glazing, encasing, marinading, pH adjustment, or a combination thereof. In addition, the mycelial food substitute or the food product is for example part of a ready-to-eat meal or a meal component.

The mycelial food substitute and the food product, respectively, of the present invention is suitable for human or animal consumption. Hence, the mycelial food substitute represents or is part of human food and/or animal feed.

### EXAMPLES

Details of the present invention are described in the following examples. The examples are not meant to limit the content of the present invention to the examples.

For texture analysis the texture analyzer Stable Micro Systems Modell “TA.XTplus” was used. The texture analyzer was operated by the software “Exponent”. The samples are prepared by cutting them having a size of 1 cm height using a cutting board, knife and a vernier calliper. Afterwards the sample is aligned underneath the test probe and the measurement is started.

### Example 1: Fungal Mycelium Treated with a Mixture of TGA and LAB for the Production of Cheese

Mycelium of Pleurotus pulmonarius generated through submerged fermentation is separated from the supernatant through filtering. The filtered solid raw mycelium is then divided into three equal parts to three cheese press form lined with a cheese cloth and pressed until no liquid separates.
a. One press form of 16.35 g is placed in a beaker and then 3 mL of tap water are added in which 0.02 g of TGase (app. 0.1 w/w % of filtrate) have been dissolved. The press form is taking up all the liquid and stored in the covered beaker at 7° C. for 24 hours.
b. One press form of 16.04 g is placed in a beaker and then 3 mL of tap water are added in which 0.1 g of ME culture (MilkySky GmbH, Alemannenweg 26, 87493 Lauben) and 0.02 g of TGase (app. 0.1 w/w % of filtrate) have been dissolved. The slice is taking up all the liquid and stored in the covered beaker at 7° C. for 24 hours.
c. One press form of 16.44 g is placed in a beaker and then 1.5 mL of tap water are added in which 0.02 g of TGase (app. 0.1 w/w % of filtrate) have been dissolved. The slice is taking up all the liquid and stored in the covered beaker at 7° C. for 24 hours. After 24 hours another 1.5 mL tap water in which 0.1 g of ME culture (MilkySky GmbH, Alemannenweg 26, 87493 Lauben) were added.
d. Vegan, sliced cheddar from Simplify (Hochland) containing as texturizer modified starch and starch is purchased and measured on texture analyzer for comparison.
e. Out of c. a 10 g piece is stored for another 35 days at 7° C. and measured on texture analyzer only.
f. Manchego, a Spanish Hard cheese (Iberico 65% Metro Deutschland) is purchased and measured on texture analyzer as a comparison.

All samples were stored at 7° C. for another 7 days, except e.

The sensory testing gave the following results:
a. Neutral, almondy aroma; neutral, slightly savory taste; texture hard
b. Sour milk aroma; slightly acidic, cheese-like taste; texture hard
c. Sour milk aroma; slightly acidic, cheese-like taste; texture hard

The texture analysis in the cutting mode yielded the following results:

| Sample | a. | b. | c. | d. | e. | f. |
|---|---|---|---|---|---|---|
| Force − | 463.46 | 399.475 | 424.2 | 416.74 | 11930.165 | 2490.376 |
| g | | | | | | |
| area − g | 928.308 | 267.176 | 398.005 | 1122.061 | 23017.561 | 9156.506 |
| per s | | | | | | |

a., b., c. are very similar in cutting force to d., which is using texturizing agents. The longer ripening period of e. is giving much higher cutting resistances, which are more similar to animal-milk protein hard cheeses such as f. compared to d. Longer ripening time and amount of added TGase influence increase the cutting resistance, so that different products such as semi-hard or hard cheese, as well as ripened meat products such as salami can be mimicked.

### Example 2: Comparison of Filtrated Vs. Shredded Mycelium

Testing the difference of shredded mycelium and filtered mycelium, both treated with TGA. Demonstrate that filet, whole-chunk can be formed only in one process (use thermomixer).

Mycelium of Pleurotus pulmonarius generated through submerged fermentation is separated from the supernatant through filtering. The filtered solid raw mycelium is then divided into three equal parts of which two are put into two cheese press form lined with a cheese cloth and pressed until no liquid separates (h. and i.).

43.55 g is placed in a thermomixer and put at level 2 in the mincing mode for 2 minutes till a homogeneous shredded mix is obtained. The 10 mL of tap water are added in which 0.04 g of TGase (app. 0.1 w/w % of filtrate) have been dissolved and blended with the mycelium. The mixture is put back into the cheese form and gently pressed to obtain the same press form as for h. and i. The press form is stored in a covered beaker at 7° C. for 24 hours.
g. 43.55 g is placed in a food blender (Thermomix T6, Vorwerk) and put at level 2 in the mincing mode for 2 minutes till a homogeneous shredded mix is obtained. The 10 mL of tap water are added in which 0.04 g of TGase (app. 0.1 w/w % of filtrate) have been dissolved and blended with the mycelium. The mixture is put back into the cheese form and gently pressed to obtain the same press form as for h. and i. The press form is stored in a covered beaker at 7° C. for 24 hours.
h. One press form of 48.25 g is placed in a beaker and then 10 mL of tap water are added in which 0.04 g of TGase (app. 0.1 w/w % of filtrate) have been dissolved. The press form is taking up all the liquid and stored in the covered beaker at 7° C. for 24 hours.
i. One press form of 40.33 g is placed in a beaker and then 10 mL of tap water are added. The press form is taking up all the liquid and stored in the covered beaker at 7° C. for 24 hours.
j. Fresh pork fillet was cooked in boiling water for 5 min. and prepared as a sample for texture measurement by cutting into a 1 cm cube.
k. Fresh chicken fillet was fried in a pan at 170° C. in a sunflower oil for 3 minutes on each side, until it showed browning on the surface.

The sensory testing yielded the following results:
g. Neutral, almondy aroma; neutral, bitter taste; texture very soft, breaking at twisting
h. Neutral, almondy aroma; neutral, slightly bitter taste; texture hard, elastic at twisting
i. Neutral, almondy aroma; neutral, slightly bitter taste; texture crumbly, immediately breaking at twisting

| | | | relative | | relative | | relative | | | Average | |
|---|---|---|---|---|---|---|---|---|---|---|---|
| | | g. | to filet | h. | to filet | i. | to filet | j. | k. | filet | Comparison |
| Cutting | Pressure − g | 331.706 | 10% | 657.448 | 19% | 235.729 | 7% | 2292.525 | 2354.479 | 3469.764 | 100% |
| | area − g | 789.817 | 17% | 1618.666 | 35% | 733.235 | 16% | 2487.076 | 4229.294 | 4601.723 | 100% |
| | per s | | | | | | | | | | |

The data show that the addition of TGase increase the cutting strength of the mycelium based food items. The lowest cutting resistance has the non-treated filter cake i. which was used as a control. Regardless the minced sample g. was much lower in cut resistance than the filtrated-only sample h. and similarly showed in sensory tasting more crumbliness and less elasticity from a whole-cut, filet like product. The comparison to the chicken and pork fillets clearly shows that the TGase treated filtrates are closer in cutting strength to the filets than the minced treated sample.

### Example 3: Fungal Mycelium for the Production of a Shrimp Substitute

Mycelium of Pleurotus pulmonarius generated through submerged fermentation is separated from the supernatant through filtering. 30 g mycelium is divided in 3 silicon-lined plastic forms with the mold resembling a half-moon and pressed firmly with a stamp until no liquid emerges. To each of the press cakes 3.5 mL of tap water is added in which 0.3 g of TGase (app. 0.1 w/w % of filtrate) have been dissolved. The press form is stored at 7° C. for 24 hours.

After 24 hours the pressed mycelium can be easily taken out of the mold without breaking. The pressed form resembles in shape a shrimp and was recognized as such by the sensory panel.

The sample l.) was fried in a pan at 170° C. in a sunflower oil for 3 minutes on each side, until it showed browning on the surface.

### Sensory Evaluation:

Aroma: fried oil, neutral; taste: savory, neutral; texture: hard, crunchy, elastic, tears at biting.

As comparison a wild-harvested shrimp (sample n.) and a vegan replacement product of sample m. (Vegan Zeastar Crispy Lemon ShrimpZ, Metro) were fried in a pan at 160° C. in a sunflower oil for 3 minutes on each side.

All three products were measured in the cutting mode with the texture analyzer yielding the following results.

| | | relative to | | relative to | | comparison |
|---|---|---|---|---|---|---|
| | l. | shrimp | m. | shrimp | n. | % |
| force g | 825.893 | 89% | 515.719 | 56% | 922.98 | 100% |
| Area g per s | 1471.295 | 58% | 1171.691 | 46% | 2533.982 | 100% |

The Mycelium filtrate sample treated with TGase shows a much more similar cutting strength relative to shrimp compared to the vegan competitive product.

### Example 4: Fungal Mycelium of Pleurotus ostreatus Var. Florida Treated with TGase

Mycelium of Pleurotus ostreatus, var. Florida generated through submerged fermentation is separated from the supernatant through filtering. The filtered solid raw mycelium is then divided into two equal parts of two cheese press forms lined with a cheese cloth and pressed until no liquid separates.
o. One press form of 48.25 g is placed in a beaker and then 10 mL of tap water are added. The press form is taking up all the liquid and stored in the covered beaker at 7° C. for 24 hours
p. One press form of 46.40 g is placed in a beaker and then 10 mL of tap water are added in which 0.04 g of TGase (app. 0.1% w/w on filtrate) have been dissolved. The press form is taking up all the liquid and stored in the covered beaker at 7° C. for 24 hours.

The sensory testing after frying yielded the following results:
o. Aroma: Sweet, yeasty aroma; taste: slightly bitter, slightly; texture: crumbly, breaking at twisting
p. Aroma: Sweet, yeasty aroma; Taste: slightly bitter; texture: hard, less flaky than Pleurotus pulmonarius

| | o. | p. |
|---|---|---|
| force g | 220.471 | 312.571 |
| Area g per s | 633.253 | 742.655 |

### Example 5: Co-Fermentation of 20% Microalgae Chlorella vulgaris with 80% Mycelium of Pleurotus pulmonarius Treated with Different Concentrations of TGase

Mycelium of Pleurotus pulmonarius is co-fermented with a suspension of 70 mL of pasteurised Chlorella vulgaris (concentration 120 g/L) in a submerged fermentation. After being separated from the supernatant through filtering, the filtered solid raw mycelium and Chlorella mix is transferred to a cheese press form lined with a cheese cloth in four equal portions and pressed until no liquid separates.
q. One press form of 41.05 g is placed in a beaker and then 10 mL of tap water are added in which 0.02 g of TGase (app. 0.05% w/w on filtrate) have been dissolved. The press form is taking up all the liquid and stored in the covered beaker at 7° C. for 24 hours.
r. One press form of 36.31 g is placed in a beaker and then 10 mL of tap water are added in which 0.04 g of TGase (app. 0.1% w/w on filtrate) have been dissolved. The press form is taking up all the liquid and stored in the covered beaker at 7° C. for 24 hours.
s. One press form of 38.42 g is placed in a beaker and then 10 mL of tap water are added in which 0.08 g of TGase (app. 0.2% w/w on filtrate) have been dissolved. The press form is taking up all the liquid and stored in the covered beaker at 7° C. for 24 hours.
t. One press form of 42.62 g is placed as a positive control with no TGase in a beaker and then 10 mL of tap water are added. The press form is taking up all the liquid and stored in the covered beaker at 7° C. for 24 hours.

To evaluate the experiment the samples were cooked for 5 minutes. Samples q, r, s turned into a more intensive white and maintained their form. Sample t disintegrated into several pieces after 2 minutes.

To test the customer experience the samples and as preparation for the sensory evaluation the samples were fried in a pan with sunflower oil at 160° C. Sample t showed intensive spattering and was breaking into several pieces. Samples q, r and s fried without significant spattering, while developing a surface browning and did not break up.

The sensory testing provided the following results with the fried products:
q. Neutral, almondy aroma; neutral, slightly savory taste; texture crumbly, slightly flaky
r. Neutral, almondy aroma; neutral, slightly savory taste; texture smooth, flaky, soft
s. Neutral, almondy aroma; neutral, slightly savory taste; texture hard, slightly flaky
t. Neutral, almondy aroma; neutral, slightly savory taste; texture crumbly

The texture analysis in the compression and cutting mode yielded the following results for the raw products:

| | q. | r. | s. | t. |
|---|---|---|---|---|
| Force - g | 345.96 | 442.559 | 658.623 | 235.729 |
| area- g per s | 953.437 | 1417.506 | 2383.509 | 733.235 |

### Example 6: Co-Fermentation of 20% Microalgae Chlorella vulgaris with 80% Mycelium of Pleurotus pulmonarius Treated with Different Concentrations of TGase

Mycelium of Pleurotus pulmonarius is co-fermented with a suspension of 70 mL of pasteurised Chlorella vulgaris (concentration 120 g/L) in a submerged fermentation. After being separated from the supernatant through filtering, the filtered solid raw mycelium and Chlorella mix is transferred to a cheese press form lined with a cheese cloth in four equal portions and pressed until no liquid separates.
u. One press form of 39.60 g is placed in a beaker and then 10 mL of tap water are added in which 0.04 g of TGase (app. 0.1% w/w on filtrate) have been dissolved. The press form is taking up all the liquid and stored in the covered beaker at 7° C. for 24 hours.
v. One press form of 36.43 g is placed in a beaker and then 10 mL of tap water are added in which 0.08 g of TGase (app. 0.2% w/w on filtrate) have been dissolved. The press form is taking up all the liquid and stored in the covered beaker at 7° C. for 24 hours.
w. One press form of 44.37 g is placed in a beaker and then 10 mL of tap water are added in which 0.2 g of TGase (app. 0.5% w/w on filtrate) have been dissolved. The press form is taking up all the liquid and stored in the covered beaker at 7° C. for 24 hours.
x. One press form of 41.45 g is placed in a beaker and then 10 mL of tap water are added in which 0.4 g of TGase (app. 1% w/w on filtrate) have been dissolved. The press form is taking up all the liquid and stored in the covered beaker at 7° C. for 24 hours.

The sensory testing cooked for 3 min. after 14 days storage at 7° C. yielded the following results:
u. Texture smooth and soft, flaky
v. Texture smooth, but slightly crumbly, slightly flaky
w. Texture hard
x. Texture very hard, firm

The texture analysis in the compression and cutting mode yielded the following results for the raw products:

| Sample | u. | v. | w. | x. |
|---|---|---|---|---|
| Force - g | 403.39 | 443.782 | 529.125 | 924.755 |
| area- g per s | 753.978 | 1186.121 | 1204.113 | 1615.211 |

The samples clearly show that also a mix of microalgae with Mycelium yields products with higher cut resistance, depending on the amount of TGase added.

### Example 7: Co-Fermentation of 20% Microalgae Chlorella vulgaris with 80% Mycelium of Pleurotus pulmonarius Treated with TGase with Added Oil and Prepared in Different Cooking Modes

Mycelium of Pleurotus pulmonarius is co-fermented with a suspension of 70 mL of Chlorella vulgaris (concentration 120 g/L) in a submerged fermentation. After being separated from the supernatant through filtering, the filtered solid raw mycelium and Chlorella mix is transferred to a cheese press form lined with a cheese cloth in three equal portions and pressed until no liquid separates.
y. One press form of 42.45 g is placed in a beaker and then 10 mL of tap water are added in which 0.04 g of TGase (app. 0.1% w/w on filtrate) have been dissolved and 1 mL of algae oil have been dispersed. The press form is taking up all the liquid and stored in the covered beaker at 7° C. for 24 hours. The product was fried on each side for 2 min in 160° C. hot rapeseed oil in a pan. After cooling the texture analysis was performed
z. One press form of 41.16 g is placed in a beaker and then 10 mL of tap water are added in which 0.04 g of TGase (app. 0.1% w/w on filtrate) have been dissolved 1 mL of algae oil have been dispersed. The press form is taking up all the liquid and stored in the covered beaker at 7° C. for 24 hours. The product was cooked for 4 min in a pan. After cooling the texture analysis was performed.
aa. One press form of 48.23 g is placed in a beaker and then 10 mL of tap water are added in which 0.04 g of TGase (app. 0.1% w/w on filtrate) have been dissolved and 1 mL of algae oil have been dispersed. The press form is taking up all the liquid and stored in the covered beaker at 7° C. for 24 hours.
bb. Vegan fish fingers (Vantastic Foods Fish Fingers, Edeka) are debreaded and fried in a pan after thawing for 2 min in 160° C. hot rapeseed oil in a pan. After cooling the texture analysis was performed.
cc. Cod filet frozen after thawing was fried on each side for 2 min in 170° C. hot rapeseed oil in a pan. After cooling the texture analysis was performed.
dd. Cod filet frozen after thawing was cooked in water for 4 min in a pan. After cooling the texture analysis was performed.

| | | relative | | relative | | | relative | | | | |
|---|---|---|---|---|---|---|---|---|---|---|---|
| | | to fried | | to cooked | | | to fried | | comparison | | comparison |
| | y. | cod % | z. | cod | aa. | bb. | % | cc. | % | dd. | % |
| force g | 565.53 | 76% | 542.14 | 94% | 543.13 | 329.864 | 44% | 573.967 | 100% | 748.083 | 100% |
| Area g | 1,164.47 | 78% | 1,415.95 | 165% | 1,600.09 | 779.356 | 53% | 857.954 | 100% | 1484.462 | 100% |
| per s | | | | | | | | | | | |

The data demonstrate that the Mycelium containing microalgae is achieving similar cutting resistance to the cooked and fried cod fish fillet indicating a greater texture similarity than the extruded competitor product.

### Example 8: Co-Fermentation of 20% Microalgae Chlorella vulgaris with 80% Mycelium of Pleurotus pulmonarius Comparing the Treatment with TGase and Different Texturizing Agents on Taste Properties of Final Product Regarding Texture and Flavor

Mycelium of Pleurotus pulmonarius is co-fermented with a suspension of 70 mL of Chlorella vulgaris (concentration 120 g/L) in a submerged fermentation. After being separated from the supernatant through filtering, the filtered solid raw mycelium and Chlorella mix is split into five equal portions of 80 g each. These samples are then treated as below and finally transferred to a cheese press form lined with a cheese cloth and pressed until no liquid separates.
ee. 0.08 g of TGase (app. 0.1% w/w on filtrate) are added, mixed and pressed. The press form is taking up all the liquid and stored in the covered beaker at 7° C. for 24 hours.
ff. 0.08 g of TGase (app. 0.1% w/w on filtrate), 0.08 g of Locust bean gum (app. 0.1% w/w on filtrate), 0.16 g of K-Carrageen (app. 0.2% w/w on filtrate) and 0.16 g of Agar (app. 0.2% w/w on filtrate) were dissolved together in 10 mL distilled water, added, mixed and pressed. The press form is taking up all the liquid and stored in the covered beaker at 7° C. for 24 hours.
gg. 0.08 g of TGase (app. 0.1% w/w on filtrate), 0.16 g of Cornstarch type 06205 (Cargill) (app. 0.2% w/w on filtrate) were dissolved together in 10 mL distilled water, added, mixed and pressed. The press form is taking up all the liquid and stored in the covered beaker at 7° C. for 24 hours.
hh. 0.08 g of TGase (app. 0.1% w/w on filtrate), 0.16 g of Cornstarch type 06214 (Cargill) (app. 0.2% w/w on filtrate) were dissolved together in 10 mL distilled water, added, mixed and pressed. The press form is taking up all the liquid and stored in the covered beaker at 7° C. for 24 hours
ii. 0.08 g of TGase (app. 0.1% w/w on filtrate), 0.08 g of Cornstarch type 06214 (Cargill) (app. 0.1% w/w on filtrate) and 0.08 g of Cornstarch typ 06205 (Cargill) (app. 0.1% w/w on filtrate) were dissolved together in 10 mL distilled water, added, mixed and pressed. The press form is taking up all the liquid and stored in the covered beaker at 7° C. for 24 hours

The resulting products are then fried at 160° C. for 2 min on each side and presented to a panel of 5 in a blind tasting. The tasting yielded the following results:
ee. Preferred sample for 3 out of 5 panelists regarding flavor and texture
ff. Least preferred sample as texture was described as soft and crumbly with some off-flavors
gg. Texture was rated soft and mushy—no panelist preferred
hh. Two panelist preferred texture due to flakiness—three panelists highlighted off-flavors
ii. No panelist preferred texture—slight off-flavor noted

The data demonstrate that alternative texturizing agents such as locust bean gum, Carrageenan, Agar or starches do not achieve the same sensory texture properties as TGase and add potential off-flavor to the Mycelium product

### Example 9: Co-Fermentation of 20% Microalgae Chlorella vulgaris with 80% Mycelium of Pleurotus pulmonarius Comparing the Treatment with TGase and Different Texturizing Agents on Texture Properties

Mycelium of Pleurotus pulmonarius is co-fermented with a suspension of 70 mL of Chlorella vulgaris (concentration 120 g/L) in a submerged fermentation. After being separated from the supernatant through filtering, the filtered solid raw mycelium and Chlorella mix is split into three equal portions of 80 g each. These samples are then treated as below and finally transferred to a cheese press form lined with a cheese cloth and pressed until no liquid separates.
jj. 0.08 g of TGase (app. 0.1% w/w on filtrate) are added, mixed and pressed. The press form is taking up all the liquid and stored in the covered beaker at 7° C. for 24 hours.
kk. 0.08 g of Locust bean gum (app. 0.1% w/w on filtrate), 0.16 g of K-Carrageen (app. 0.2% w/w on filtrate) and 0.16 g of Agar (app. 0.2% w/w on filtrate) were dissolved together in 10 mL distilled water, added, mixed and pressed. The press form is taking up all the liquid and stored in the covered beaker at 7° C. for 24 hours. Then the sample is submerged vacuumed in a plastic bag for 5 min at 90° C. to ensure gelling of texture additives.
ll. 0.24 g of TGase (app. 0.3% w/w on filtrate) were dissolved in 10 mL distilled water, added, mixed and pressed. The press form is taking up all the liquid and stored in the covered beaker at 7° C. for 24 hours.

The texture analysis in the compression and cutting mode yielded the following results for the raw products.

| Sample | jj. | kk. | ll. |
|---|---|---|---|
| Force - g | 782.02 | 287.61 | 975.74 |
| area- g per s | 2324.84 | 986.42 | 2846.12 |

The data demonstrate that the Mycelium containing microalgae is achieving a higher and adjustable cutting resistance compared to texture modifiers making it more similar to animal whole-cut products

1. Method for the production of a whole-cut mycelial food substitute having fibrous structure consisting of
a) culturing a fungus in growth medium to form a mycelium,
b) harvesting the mycelium of the fungus for example by filtration, centrifugation, pressing, screening, evaporation or a combination thereof to isolate the mycelium of the fungus from the growth medium,
c) adding a cross-linking enzyme to the harvested mycelium of the fungus, wherein hypha of the mycelium are cross-linked, and
d) optionally bringing the linked hypha in a desired form before or after step c), and/or adding an additional component such as a flavoring agent after step c)

2. Method according to claim 1, wherein in step a) an algae, bacteria, archaea, animal cells or a combination thereof is cultured together with the fungus, wherein an algae, bacteria, archaea, animal cells or a combination thereof is added to the harvested mycelium of step b) and/or wherein an algae, bacteria, archaea, animal cells or a combination thereof is added to cross-linked hypha of step c).

3. Method according to claim 1 or 2, wherein the harvested mycelium of step b) is brought into a desired form before the cross-linking enzyme is added to the harvested mycelium in step c).

4. Method according to any one of claims 1 to 3, wherein the cross-linking enzyme is selected from the group consisting of a laccase, peroxidase, tyrosinase, sortase, lysyl oxidase, amine oxidase, transglutaminase or a combination thereof.

5. Method according to any one of claims 1 to 4, wherein the crosslinking enzyme is present in a concentration of about 0.01 to 5%, about 0.1 to 3% or about 1 to 2%.

6. Method according to any one of claims 1 to 5, wherein in optional step d) a non-animal fat, flavoring, coloring, preservative, texture modifier, a processing aid or a combination thereof is added to the harvested mycelium before cross-linking of the hypha of the mycelium, and/or
a non-animal fat, non-animal protein, flavoring, coloring, preservative, texture modifier, a processing aid or a combination thereof is added to the harvested mycelium after cross-linking of the hypha of the mycelium.

7. Method according to any one of claims 1 to 6, wherein the fungus is selected from the division basidiomycota, ascomycota or a combination thereof.

8. Method according to any one of claims 2 to 7, wherein the bacteria is selected from the group consisting of acetobacter, arthrobacter, bacillus, bifidobacterium, brachybacterium, brevibacterium, carnobacterium, corynebacterium, enterococcus, gluconacetobacter, gluconobacter, hafnia, halomonas, kocuria, komagataeibacter, lactobacillus, lactococcus, leuconostoc, macrococcus, microbacterium, micrococcus, oenococcus, propionibacterium, staphylococcus, streptococcus, streptomyces, tetragenococcus, weissella, zymomonas or a combination thereof.

9. Method according to any one of claims 1 to 8, wherein the algae is selected from the division chlorophyta.

10. Whole-cut mycelial food substitute having fibrous structure obtainable by a method according to any one of claims 1 to 9.

11. Whole-cut mycelial food substitute having fibrous structure comprising a fungal mycelium and optionally bacteria, algae, archaea, animal cells or a combination thereof, wherein hypha of the mycelium are cross-linked.

12. Whole-cut mycelial food substitute according to claim 10 or 11, wherein the cross-linked hypha form fibers having a length of about 1 to 50 mm, about 2 to 30 mm or about 3 to 20 mm, and optionally a thickness of about 1 to 500 mm, about 3 to 450 mm, about 5 to 400 mm, about 8 to 350 mm, about 10 to 300 mm, about 15 to 250 mm, about 20 to 200 mm, about 30 to 150 mm or about 50 to 100 mm.

13. Whole-cut mycelial food substitute according to any one of claims 10 to 12 having a cut resistance of about 250 to about 15000 g or of about 400 to about 5000 g, for example wherein the area is of about 600 to about 30000 g sec or about 800 to about 15000 g sec. and optionally a water content of about 5 to 98%, about 10 to 90%, about 15 to 80%, about 20 to 70%, about 30 to 60%, about 40 to 50 or about 5 to 20%.

14. Use of a whole-cut mycelial food substitute according to any one of claims 10 to 13 for the production of a food product.

15. Food product comprising a whole-cut mycelial food substitute according to any one of claims 10 to 13 and an edible for example selected from the group consisting of flavoring agents such as spices, colorings, processing aids, conserving aids, plant-based proteins, fats and a combination thereof.
